(19) United States
(12) Patent Application Publication
(10) Pub. No.: US 2025/0066988 A1
(43) Pub. Date: Feb. 27, 2025

(54) Multiple Garment and Functions Processing Apparatus

(71) Applicant: Mukana Mpanzu Muanda, Pearland, TX (US)

(72) Inventor: Mukana Mpanzu Muanda, Pearland, TX (US)

(21) Appl. No.: 18/814,561

(22) Filed: Aug. 25, 2024

Related U.S. Application Data
(60) Provisional application No. 63/578,671, filed on Aug. 25, 2023.

(51) Int. Cl. D06F 67/10; D06F 39/02; D06F 43/00; D06F 60/00; D06F 103/02; D06F 105/40

(52) U.S. Cl. CPC D06F 67/10; D06F 39/022; D06F 43/007; D06F 60/00; D06F 2103/02; D06F 2105/40

(57) ABSTRACT

A garment processing unit is provided that can carry out ironing, deodorizing, screen printing, dry cleaning, wet vacuuming, steam cleaning and the like over multiple garments simultaneously. The unit delivers heat and/or steam, air, water, detergents and fragrances to clothing, garment or fabrics and the like for removing wrinkles and or applying heat transfer, cleaning, drying, deodorizing, and perfuming to single or multiple articles of clothing simultaneously.

### CROSS-REFERENCE TO RELATED APPLICATION

This application claims the benefit of U.S. Provisional Patent Application No. 63/578,671, filed Aug. 24, 2023, which is incorporated by reference herein in its entirety.

### FIELD OF DISCLOSURE

The present invention relates to a garment processing system and device for the processing of multiple articles of clothing and other articles made of a garment or fabric. More particularly, the present invention relates to a unit that delivers heat and/or steam, air, water, detergents and fragrances to clothing, garment or fabrics and the like for removing wrinkles and or applying heat transfer, cleaning, drying, deodorizing, and perfuming to single or multiple articles of clothing simultaneously. It can also process ink or heat to transfer patterns or graphics onto fabrics.

### DESCRIPTION OF RELATED ART

In the home or commercial laundry, the processing of garments typically uses a washing machine, to wash the garments by completely soaking the garments. The machines wash garments with a considerable amount of water consumption. A separate device is separately used to dry the garments that have been washed. The drying cycle time and energy required is dependent of the weight of garments and the water that has to be removed. Additionally, a single heating device is used for applying steam, dry steam and heat or air, to remove wrinkles, and to apply heat transfer to the fabrics.

In such applications, steam and or heat is applied to a garment through a single device at a time that a user applies to the fabric. In recent advancements there have devices created such as spot cleaners, garment refreshing system, stain removers, handheld steamers and more. However, there are limitations when it comes to combining wash, dry and press or other processes into one single tool reducing handling, time, energy and water consumption while improving ergonomics. In situations where it is desired to process several items in a discrete manner (not in batch), such known designs have limited results, applications and functionalities.

In the garment printing, dry-cleaning, laundry and ironing business, the processing of garments typically uses a single heating device at a time for applying steam and heat or air, to remove wrinkles, and to apply heat transfer to the fabrics. This specific apparatus allows for the following functionalities for single and/or simultaneous garment processing: removing wrinkles, deodorizing, perfuming garments, cleaning garments, drying garments and applying graphics (designs) on garments. Garments are processed in a discrete manner (not in batch) meaning that each garment is processed in its own station allowing the process to be customized per garment if needed. Through simultaneous processing of multiple garments, the processing rate of this apparatus exceeds the limitations of the current established practices. It allows for single, multiple or simultaneous users' configurations as well.

### SUMMARY

The present disclosure provides the ability to iron multiple garments of clothing at the same time. The ironing is obtained by moving the arm using a 3-axis manual or automated machine or robot. The single extended and extendable arm is attached to the 3-axis machines on which the multiple devices are attached. The heating devices are placed in such a way that vibration or flection is controlled, and balance is obtained. The heating devices connect to a plate of high thermal conductivity. The plate mat be made of aluminum.

This specific collaborative apparatus allows multiple garment processing at the same time via robotic or manually control arm, the combination of functionalities via a single tool adapted in a single workstation therefore improving the ergonomics or human factors adaptation. Garments are supported by a support that can be porous or non-porous and water resistant and it reduces the water consumption used to wash as the washing of the garment does not have to be done on the entirety of the garment thus reducing effort for drying the items. The functionalities are removing wrinkles, deodorizing, perfuming garments, cleaning garments, applying fragrances, T-shirt printing, and more.

In another exemplary embodiment this invention has an adaptable mechanism that enables a combination of functions such washing pressing or ironing or another garment care into a single tool. This mechanism can adapt an iron, a wet vacuum cleaner, jet cleaning air dryer, dry steamer, ultrasonic cleaning and more for the purpose of facilitating garment care. The mechanism is comprised of a method to mount onto the extended arm of the machine. It also channels the hosing, piping or tubing of devices that are combined for the purpose of processing garment these devices are but not limited to wet vacuum system, air-dryer, dry steamer, jet cleaner, ultrasonic, detergent or fragrance applicators and more.

In another exemplary embodiment, tee shirt or garment printing, a plate of high thermal conductivity is added and used to provide pressure and heat. The pressure is obtained by driving an arm down using a 3-axis manual or automated 3-axis machine or robot. The single extended and extendable arm is attached onto the 3-axis machines on which the multiple devices are attached. The devices are placed in such a way that vibration or flection is controlled, and the system is balanced.

In yet another embodiment a fixture (removable) is added to a plate (of high thermal conductivity) allow washing and drying garment using wet vacuum cleaning type (spot cleaners) system. The nozzle of the wet vacuum system will be mounted with a mesh or other material allowing fluids with enough tension to refrain the garment from being displaced by pressure. This allows low water consumption per garment vs standard rotary garment washing.

In yet another embodiment, a fixture (removable) is added to a plate (removable) to allow washing and drying of the garment using hot air vacuum system and dry-cleaning powder/scouring (already available in the industry) to dry clean garments. This is an environmentally friendly alternative to solvent dry cleaning.

In yet another embodiment, a fixture is added to aluminum plates (removable) to allow a screen-printing squeegee to ink and apply graphics to garment.

The extendable arm in this embodiment allows for multiple devices to be mounted and therefore simultaneously process garments. The metal plates have a high thermal conductivity used for heat transfer. The metal plate serves as a fixture on which to mount the other fixtures allowing multiple functionalities. The metal plates have a high thermal conductivity used for heat transfer. The metal plate serves as a fixture on which to mount the other fixtures allowing multiple functionalities.

These and other aspects, objects, features, and embodiments will become apparent to a person of ordinary skill in the art upon consideration of the following detailed description of illustrative embodiments exemplifying the best mode as presently perceived.

### BRIEF DESCRIPTION OF THE DRAWINGS

For a more complete understanding of the embodiments and the advantages thereof, reference is now made to the following description, in conjunction with the accompanying figures briefly described as follows:

FIG. 1 is an illustration of a perspective view of a garment proceeding unit according to the present disclosure; this depicts a view of a station for one garment, according to the present invention.

FIG. 2 is an illustration of a second perspective view of the garment processing unit, according to the present invention.

FIG. 3 is an illustration of a perspective view of the garment processing unit in a tethered state, according to the present invention.

FIG. 3A is a picture of the garment processing unit configuration in the tethered state, according to the present invention.

FIG. 4 is an illustration of a perspective view of a heating device (iron) with the metal plate attachment, according to the present invention.

FIG. 5 is an illustration of a second perspective view of a heating device (iron) with the metal plate attachment, according to the present invention.

FIG. 6 is an illustration of a perspective view of the garment processing unit in a tethered state with the metal plate attachment, according to the present invention.

FIG. 6A is a picture of prototype of a perspective view of a garment processing unit in its tethered state according with the metal plates, according to the present invention.

FIG. 7 is an illustration of a perspective view of the table to accommodate different sized garments, according to the present invention.

FIGS. 8 and 9 is an illustration of a perspective view of a table capable of being turned and rotated, according to the present invention.

FIG. 10 is an illustration of the extended table in a tethered state to accommodate multiple stations, according to the present invention.

FIG. 11 is picture of prototype of a prototype of a wet vacuum system mounted on the plate through a quick-change mechanism allowing washing and drying of garments, according to the present invention.

FIG. 12A depicts an attachment of an activated charcoal filter cartridge mounted on the suction/spray nozzle (in the flow path) of the air/vent, according to the invention.

FIG. 12B depicts an attachment of a sponge (soaked with detergent) mounted on the suction/spray nozzle, according to the invention.

FIG. 12C depicts a vacuum cleaner similar to FIG. 11, however instead of liquid water being used, steam or hot air is used to activate a powder dry cleaning detergent to allow cleaning delicate (dry cleaning only) garments, according to the present invention.

FIG. 12D illustrates how the use of sensors and existing methods (visual, gas meters, ammonia meters, cameras, ph. meters) are used to detect where a garment needs to be cleaned, according to the present invention.

FIG. 13 is an illustration of a screen print squeegee mounted on the plate through a quick-change mechanism allowing processing of ink to deliver designs or graphics on garments, according to the present invention.

FIG. 14 depicts how the available working area in this present invention can be much larger than the original area in the 3-axis machine, according to the present invention.

FIG. 15 is an illustration of a prototype of a roller mounted on the plate through a quick-change mechanism allowing the delivering of a fragrance or perfume on garments, according to the present invention.

FIG. 16 is an illustration of the garment processing unit processing multiple types of garments, according to the present invention.

FIG. 17 is an illustration of the garment processing unit having a combined configuration, according to the present invention.

FIG. 18 is an illustration of the combined garment processing unit with the heating element in contact with the garment and the arms actuated, according to the present invention.

FIG. 19 is an illustration of the combined garment processing unit with the vacuum system in contact with the garment, according to the present invention.

FIG. 20 is a flow diagram for operation, according to the invention.

The drawings illustrate only example embodiments and are therefore not to be considered limiting of the scope described herein, as other equally effective embodiments are within the scope and spirit of this disclosure. The above objects, features and advantages of the present disclosure will the apparent and understood by those skilled int the art from the following detailed description, drawings, and accompanying claims. As shown throughout the drawings, like reference numerals designate like or corresponding parts.

### DETAILED DESCRIPTION

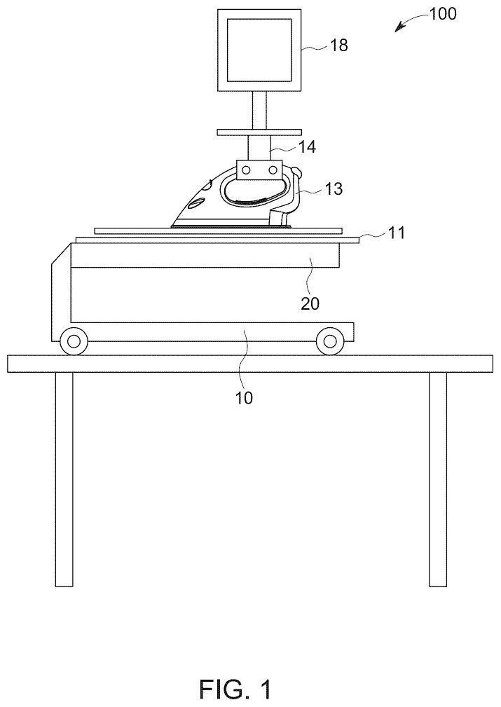

A garment processing unit 100 according to an embodiment of the present invention is shown in FIG. 1. Garment processing unit 100 includes a table 10 having an elongated shelf 20 having insulation 11 thereon. Insulation 11 is used to provide padding and to prevent scalding of the elongated shelf 20. The heating element 13 is suspended above table 10 and controlled by movement of beam 18. The beam 18 is used as support for the device or devices and serves as well for pipe and cable management. We can also imagine a configuration where the garment unit is mobile around beam 18. This can be achieved through a 3-axis machine underneath/turntable conveyor systems or automated guided vehicles. We add this precision to emphasize that it is the size of the extended and extendable arm is what is allowing to make this methodology practical in terms of output rate.

In the figures heating element 13 is depicted as an iron, however the heating element can be any suitable heating device to heat the garment for ironing, heat transfer and the like. Heating element 13 is coupled to beam 18 by coupling mechanism 14 (adjustable in height and with another mechanism to allow

for small side by side motion to realign in case of entanglement or excessive force). Coupling mechanism 14 has adjusting bolts 15. Adjusting bolts 15 can be raised or lowered to make fine adjustments to the height of heating element 13 to add pressure to the garment placed on insulation 11 that is to be processed. These fine adjustments can be used to de-wrinkle the garment by concentrating the heated portion of the heating element 13 to the garment. Heating element 13 can be of which is generally larger in size and heavier than residential types of irons. In this present invention garments are laid in such a way that the surface is as even as possible to avoid entrapment and collision of the machine. To be more specific the shirts are put inside out so that the pockets and buttons are facing down. The same strategy is used for pants or other types of items. Addons are also used to even out the surface. The ironing device is moving on the surface that is the most even.

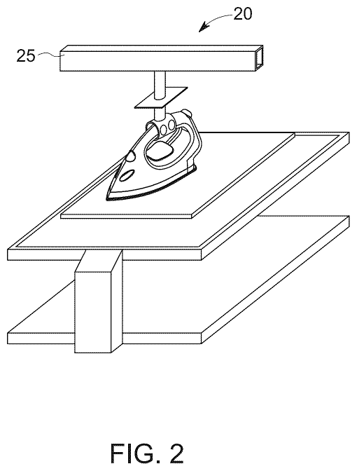

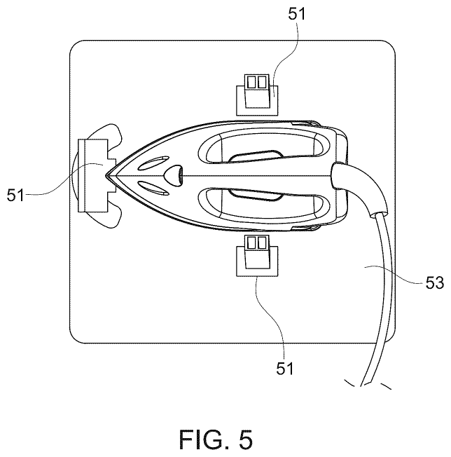

As will be discussed in more detail, processing of multiple garments using garment processing unit 100 can be accomplished by movement of the heating devices across the surface of the garment in a tethered state, as shown in FIGS. 2-5. As shown in FIG. 2, beam 18 has channel 25, wherein beam 18 is coupled to arm 26 via channel 25 to facilitate the de-wrinkling and ironing of the garment that has been positioned on the elongated shelf of the table 10 and insulation 11.

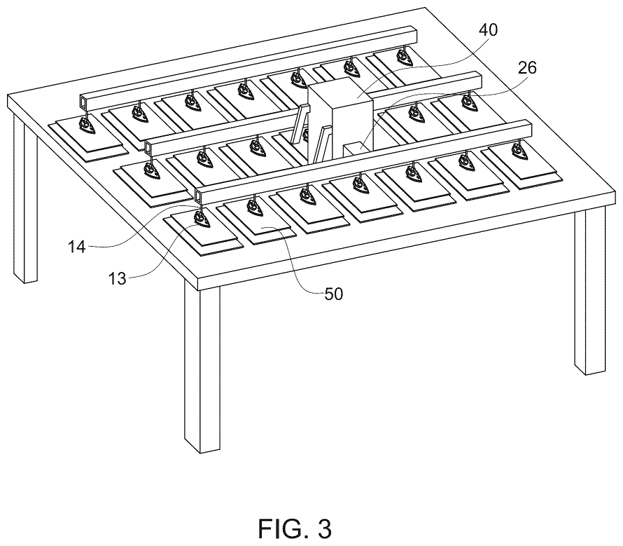

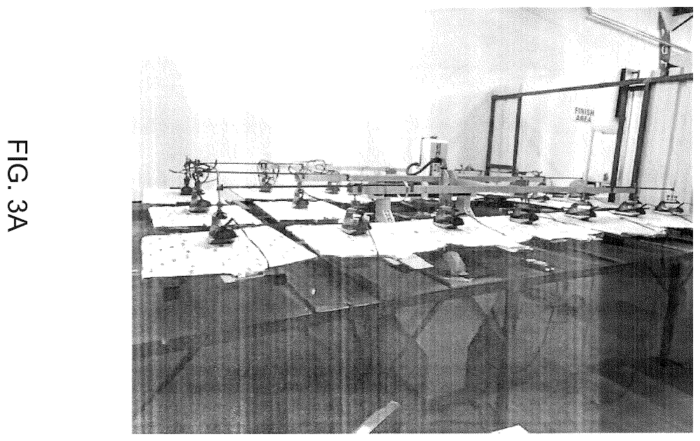

FIG. 3 depicts the tethered state of multiple processing units 100 all coupled together and by movement arm 26 by machine 40. A computer processor can facilitate the movement of the arm 26 in both the X, Y and Z directions. Arm 26 is made of a rigid material such as steel. Using the multiple processing units 100 the ironing of multiple garments can occur simultaneously.

The multiple garment process can be managed by a computer program through 3-axis machine or can manually be managed through a manual or semi manual 3-axis machines. The programs are made in such way that machine is instructed to cover the entire surface area of the garments. Garments can be repositioned where necessary.

FIG. 3A. shows the operation of the garment processing unit in the tethered state.

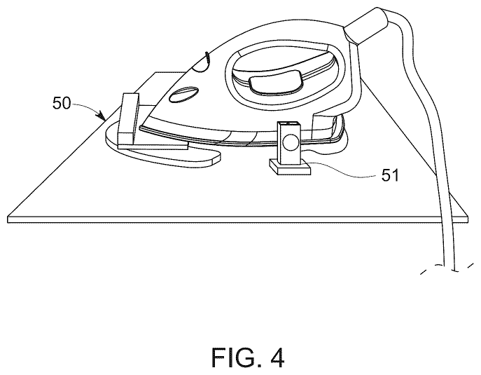

In another embodiment, as shown in FIGS. 4-6, a metal plate 50 can be coupled to heating element 13 to facilitate the application of a thermal transfer item to a garment. The heating element 13 can be coupled using clips 51. Heat from the heating element 13 can be transferred to metal plate 50 to heat the metal to a temperature suitable melt the resin on heat transfer items. The iron max temperature limit has been increased to account for heat loss through plate. The metal plate 50 can be made of aluminum or any other metal suitable to heat the resin to a proper temperature to melt the resin. Side walls can be added to the metal plate for special vertical heat transfer needs.

To carry out the present invention a hot-melt thermoplastic resin (through a transfer sheet) is formed on the back of a thermal transfer item. Heat from the metal is then used to melt the hot-melt resin and thus melt the applique, adhering the applique to the fabric.

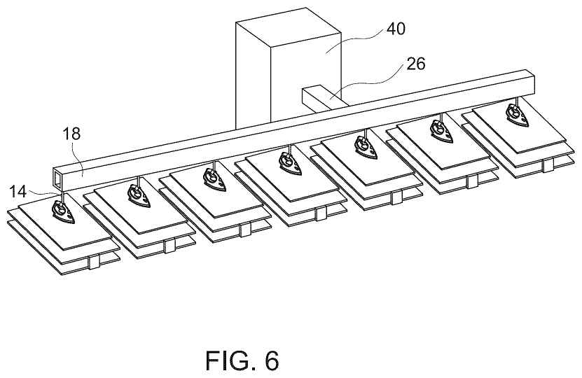

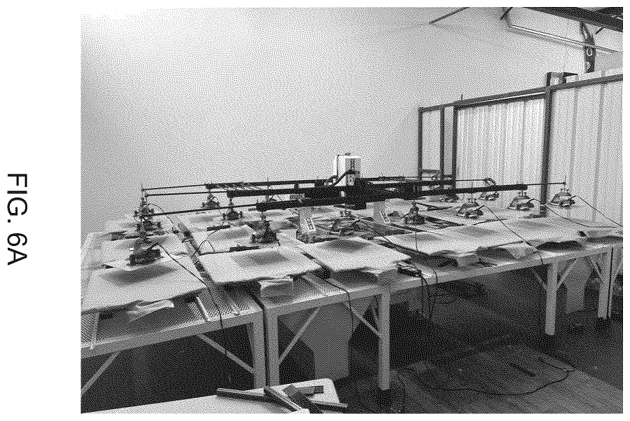

Similar to FIG. 3, FIG. 6 depicts tethered state of multiple processing units 100 having the metal plates 50 attached thereto. Movement is accomplished as described with respect to FIG. 3 above.

In FIG. 6A, is an illustration of a perspective view of a garment processing unit base shown in FIG. 1 according to the present disclosure in its extendable state. It shows that the unit can accommodate various garments sizes.

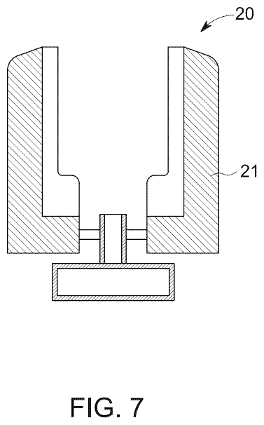

FIG. 7 is an illustration of the elongated shelf 20 according to the present invention. The elongated shelf 20 can have extendable portions 21 that will provide a wider surface so that larger garments and or rugs can be placed on the table 10. Garments are mounted on garment shelf is made of water and heat-resistant material but not limited to things such as granite, carbon fiber, glass and more.

The table 10 is designed such that it can present a front and back side to the processing tool by a flipping mechanism. A meshed sleeve can be applied around garment processing unit with the purpose or secure garments parts such as shirt sleeves. The mesh material allows processing garments even though the sleeve is around it. The support allows a garment to be kept in place as vacuum pressure is applied. In yet another embodiment, the mesh material can allow for processing and fluid can flow from one side of the garments to the other for the purpose of processing the garments.

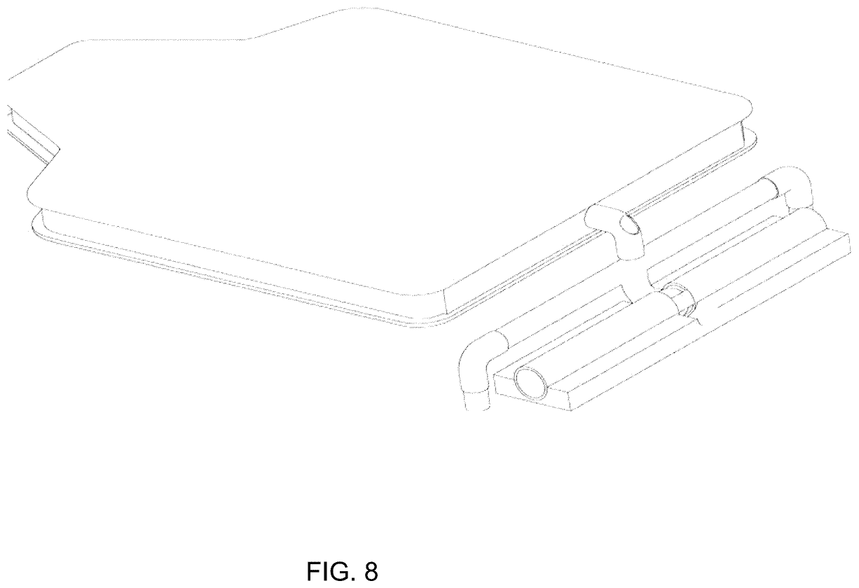

FIG. 8 is a perspective view of the garment processing unit according to the present invention that can accommodate a large number of garments. In this case 18 garments can be processed.

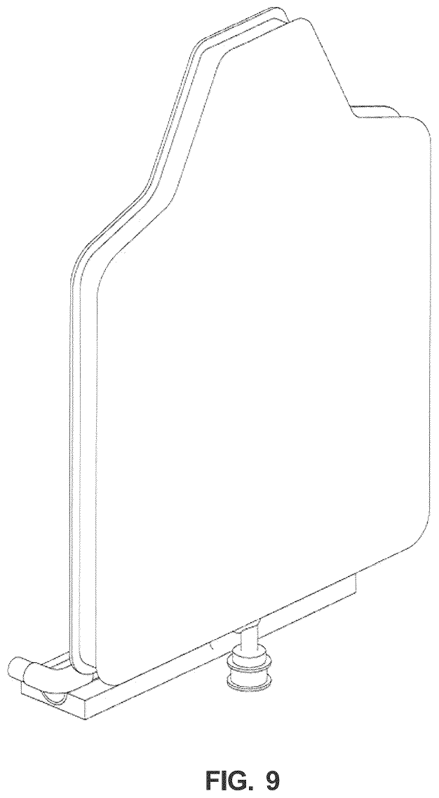

FIGS. 9-11 illustrate alternate embodiments that the present invention can be used for.

FIG. 9 is an illustration where the garment processing unit 100 of the present invention is adapted to clean various garments such as: shirts, pants, dresses and more; using a wet vacuum system mounted on the metal plate 50 allowing the washing and substantially drying of the object.

Here, vacuum hose 45 is attached to the metal plate 50 using a quick release mechanism 51. The vacuum hose 45 connected to suction/spray nozzles 60. Suction/spray nozzles 60 can spray cleaning solution and water on the garments and subsequently extract the liquid from the garments by applying suction through vacuum hose 45.

In another embodiment, FIG. 9A illustrates the attachment of an activated charcoal filter cartridge 70 mounted to the suction/spray nozzle 60 and in the flow path and very close to the garment. This creates a deodorizing tool. Here, the charcoal filter cartridge of the vacuum is placed into the air flow thus to cleaning and deodorizing the garment. In another embodiment, FIG. 9B illustrates an embodiment where the attachment of a sponge 75 (soaked with detergent) and mounted to the suction/spray nozzle 60 in such a way that the sponge 75 is used to put detergent on the garment and the vacuum extracts the solution. Using this method, a user can implement another wet vacuum cleaning method.

In an alternate embodiment FIG. 9C depicts a garment processing unit similar to FIG. 9, however instead of liquid water being the fluid, steam or hot air is used to activate powder dry cleaning detergent to allow cleaning delicate (dry cleaning only) garments. This allows the user to implement a dry-cleaning method. In still yet another embodiment, FIG. 9D illustrates how the use of human operator or already existing sensors (visual, gas meters, ammonia meters, cameras, ph. meters) are used to detect where a garment needs to be cleaned. In this present invention, unlike current rotary washing machine and dry-cleaning machines, it is possible to detect various existing methods (visual, gas meters, ammonia meters, cameras, ph. meters) smell, dirt, stain and select a path of cleaning tool to focus only (or more) in the area that needs the most cleaning. This process further reduces the consumption of water and detergent. Thus, making this present invention an improvement to existing environmental solution. The reduced water consumption eliminates the need. These sensors detect where a garment is soiled and the 3-axis machine can then position the cleaning method used over that area, actuate the cleaning method and clean the area.

For example, using this method, if during a scan of the garment, the ammonia sensor detects that the arm pit area of the garment is soiled, then the S4 and S6 areas of the garment needs to be cleaned or needs extra attention and other areas of the garment either doesn't need attention or needs less attention. For the garment cleaning applications of this system the control program can be planned as to cover the whole area of the garment on the station (front and back). The programs can also be selected in such way where an algorithm is created to where a smell detector (can be run through garments and determine areas that will need to be washed) This detection will then allow to call for sub programs that will represent movement of the cleaning device over that sub area this allow to reduce the time but more importantly to address the area that needs the cleaning the most leaving a better quality and reducing even more the water (fluid) consumption making the system and this method a good environmental alternative.

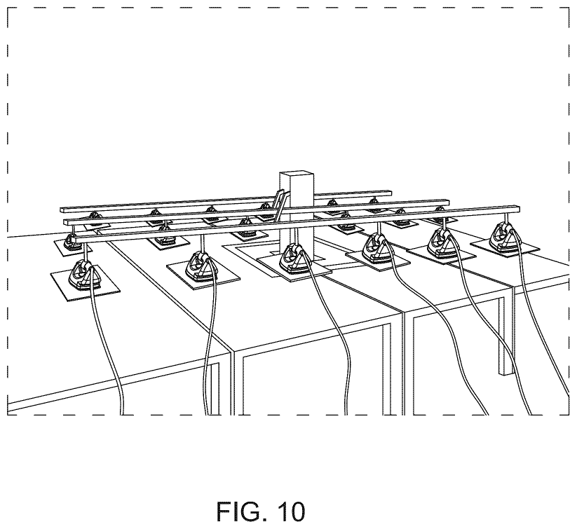

In yet another embodiment, FIG. 10 is an illustration of a screen printing method according to this invention. Here, a squeegee 100 is mounted on plate 50 through a quick-change mechanism, allowing processing of ink to deliver designs or graphics on multiple garments according to the present invention. The 3-axis machine can then position the squeegee to put the layers of ink on the garment. The screen-printing method used is already known in the art.

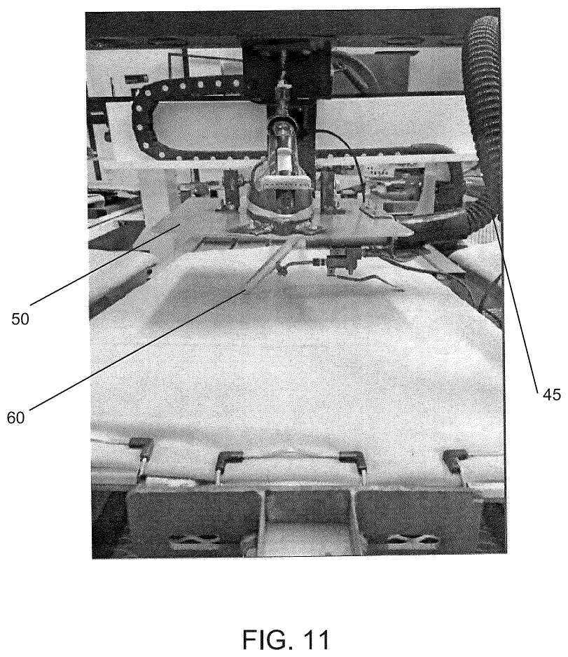

FIG. 11. illustrates how the available working area in this present invention can be much larger than the original area with the 3-axis machine 40. The extendable arm 26 extends out to an area much larger than the area of the machine. That area is depicted in the drawing on the table that supports the various garment stations. This extended arm in embodiment could be on a robot arm.

This arm is attached to the effector and to the distal end of the arm, a standard 3D machine can carry movement of the 3D robotics machine (in this configuration) into up to 18 other work envelopes for garment processing. Computer numerical control (CNC) is a manufacturing method that automates the control, movement and precision of machine tools using preprogrammed computer software. Up to 18 multifunctional effectors are then attached to that arm to carry laundry or garment processing applications. This is a more efficient option than buying 18 machines to perform the same task or having one 3D CNC machine or another dedicated machine that covers 18 times the original area.

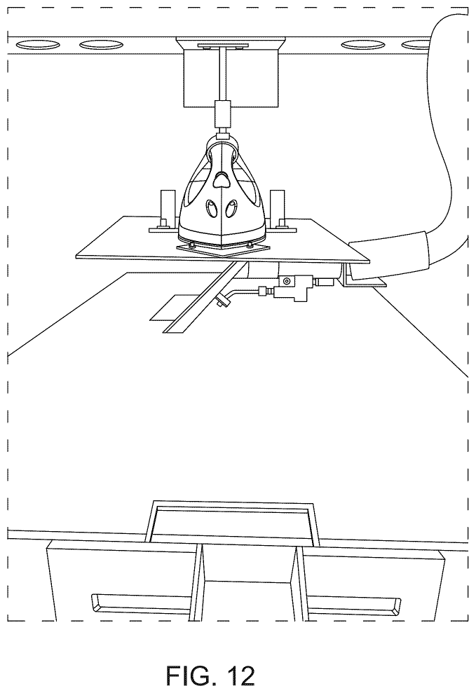

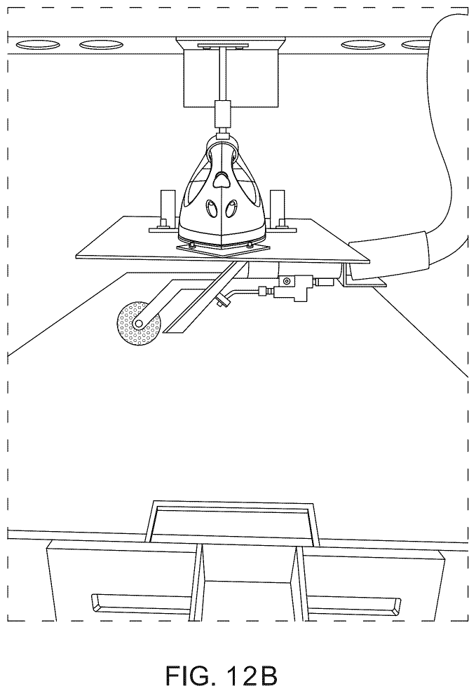

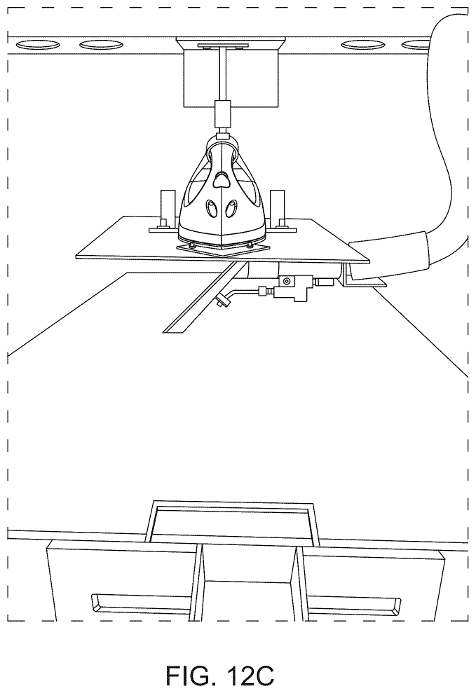

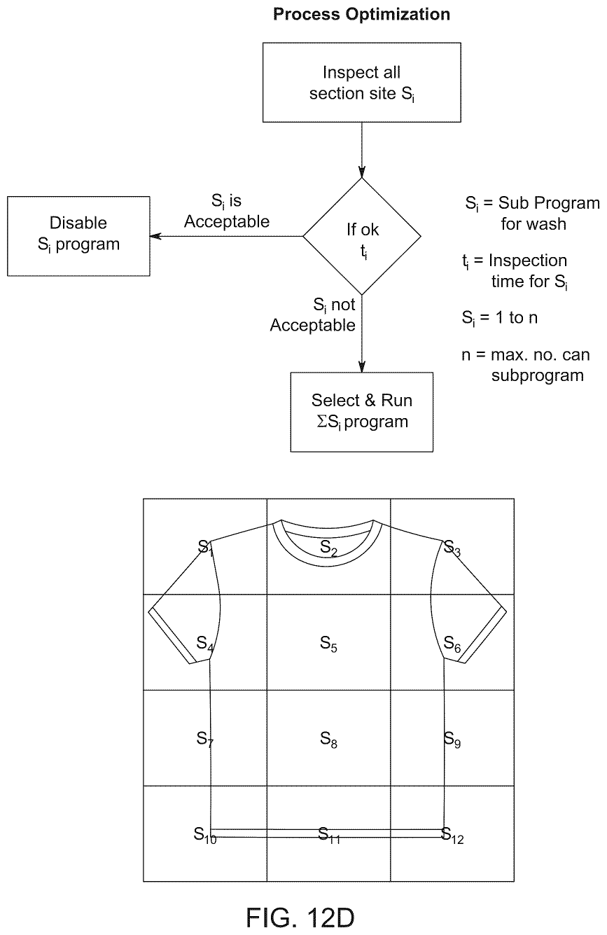

FIG. 12 illustrates a roller mechanism 105 for deodorizing a garment.

FIG. 12A depicts an attachment of an activated charcoal filter cartridge mounted on the suction/spray nozzle (in the flow path) of the air/vent according to the invention.

FIG. 12B depicts an attachment of a sponge (soaked with detergent) mounted on the suction/spray nozzle according to the invention.

FIG. 12C depicts a vacuum cleaner similar to FIG. 11, however instead of liquid water being used, steam or hot air is used to activate a powder dry cleaning detergent to allow cleaning delicate (dry cleaning only) garments.

FIG. 12D illustrates how the use of sensors and existing methods (visual, gas meters, ammonia meters, cameras, ph. meters) are used to detect where a garment needs to be cleaned.

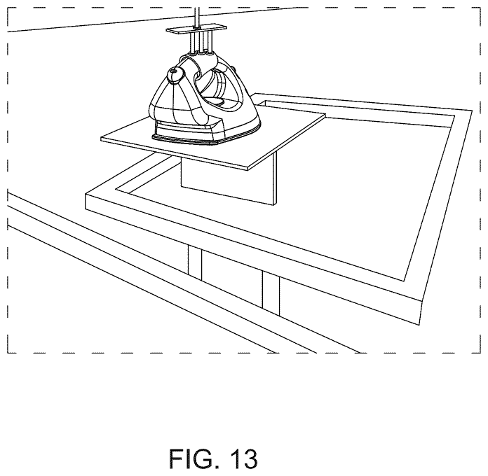

FIG. 13 illustrates how a screen print squeegee is mounted on the plate through a quick-change mechanism allowing processing of ink to deliver designs or graphics on garments according to the present invention. This process is a screen printing is a process where ink is forced through a mesh screen onto a surface. Making certain areas of the screen impervious to printing ink creates a stencil, which blocks the printing ink from passing through the screen. The ink that passes through forms the printed image. Screen printing is a printing technique where a mesh is used to transfer ink (or Dye) onto a substrate, except in areas made impermeable to the ink by a blocking stencil. A blade or squeegee is moved across the screen to fill the open mesh apertures with ink, and a reverse stroke then causes the screen to touch the substrate momentarily along a line of contact. This causes the ink to wet the substrate and be pulled out of the mesh apertures as the screen springs back after the blade has passed. One color is printed at a time, so several screens can be used to produce a multi-colored image or design.

Traditionally, silk was used in the process. Currently, synthetic threads are commonly used. The most popular mesh in general use is made of polyester. There are special-use mesh materials of nylon and stainless steel available to the screen-printer. There are also different types of mesh size which will determine the outcome and look of the finished design on the material.

The technique is used not only for garment printing but for printing on many other substances, including decals, clock and watch faces, balloons, and many other products. Advanced uses include laying down conductors and resistors in multi-layer circuits using thin ceramic layers as the substrate.

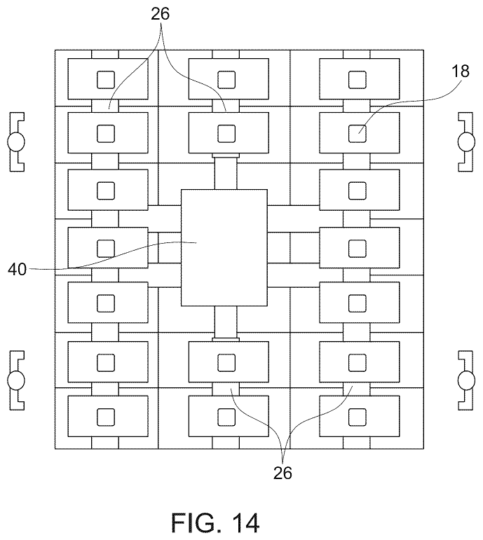

FIG. 14 depicts how the available working area in this present invention can be much larger than the original area in the 3-axis machine. By tethering the tables 10 (as discussed above) together you can create a larger working area. Arms 26 move unit 18 across the garments surface area using machine 40.

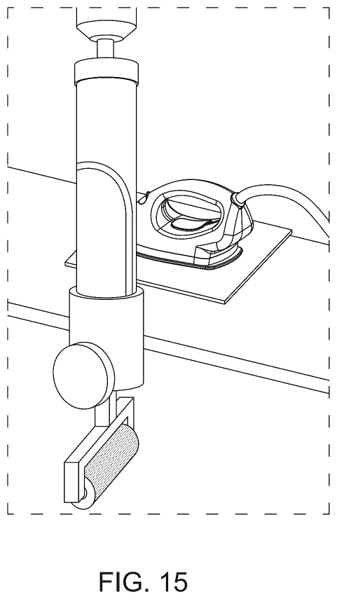

FIG. 15 is depicts a roller mounted on the plate through a quick-change mechanism allowing the delivering of a fragrance or perfume on garments. This roller can be made of any permeable material that essentially holds a fragrance.

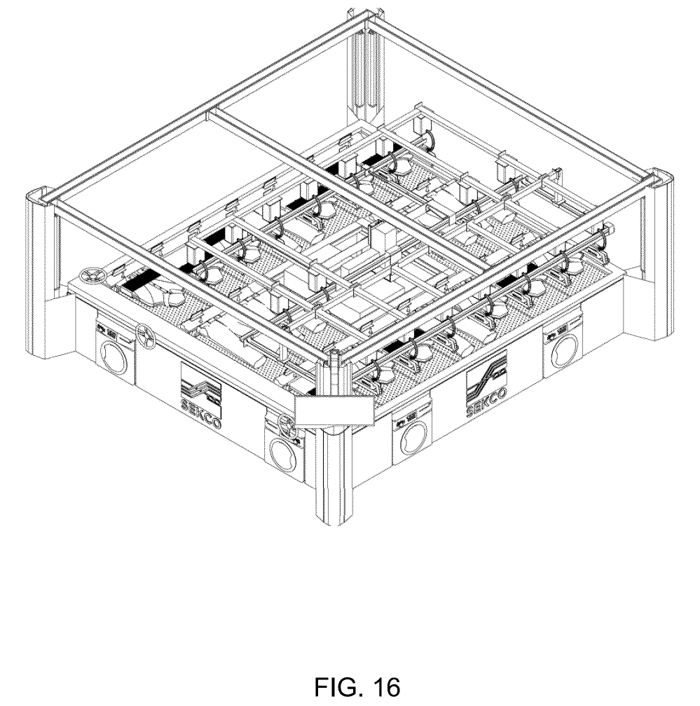

FIG. 16 depicts the garment processing unit handling the processing of multiple types of garments. As one can see shirts can be processed on the first row, pants can be processed on the second row and shirts can be processed on the third row or any combination as desired.

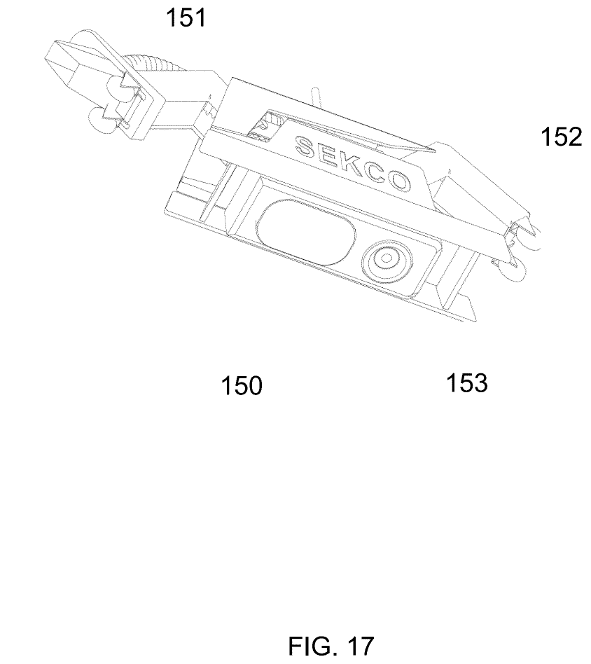

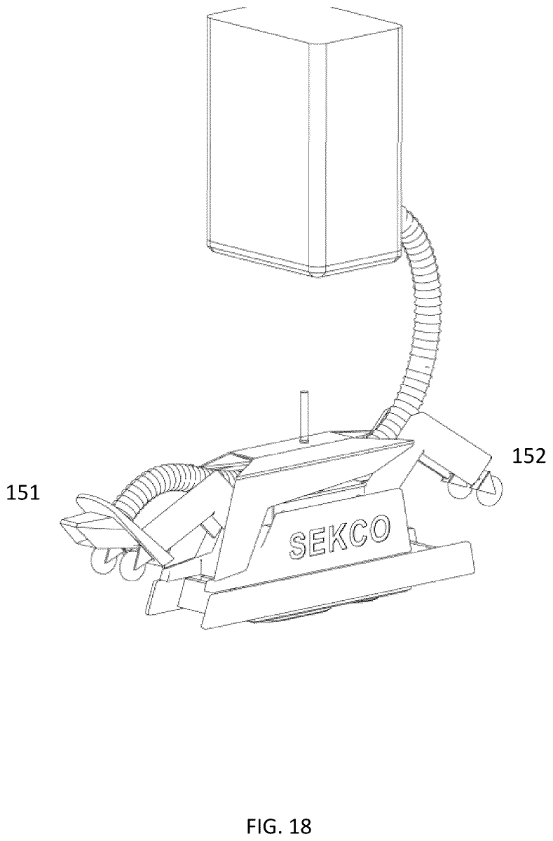

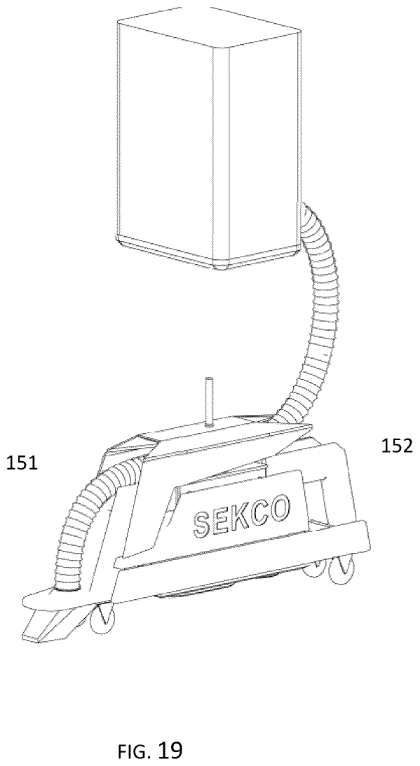

FIGS. 17, 18 and 19 are illustrations of the garment processing unit having a combined configuration. Here the garment processing unit may have a heating element 150 located on the bottom to be near a garment. The unit may have arms 151, 152 that acuate to bring and a wet vacuum system close to the garment. The wet vacuum system can spray a solution on the garment and the vacuum may extract the solution from the garment. The unit may also have a ph sensor or stain detector 153 or the like to indicate where the garment needs to be cleaned.

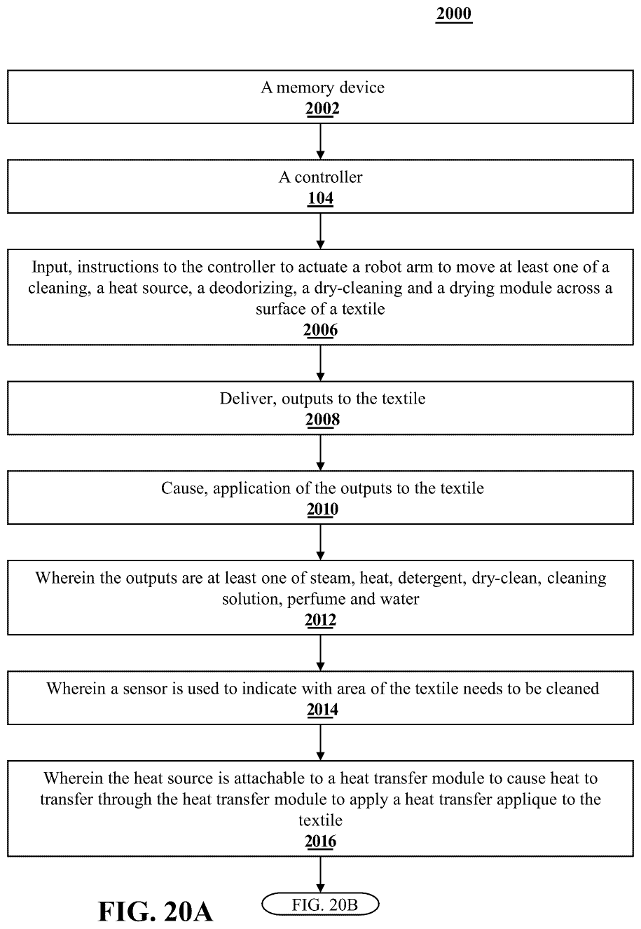

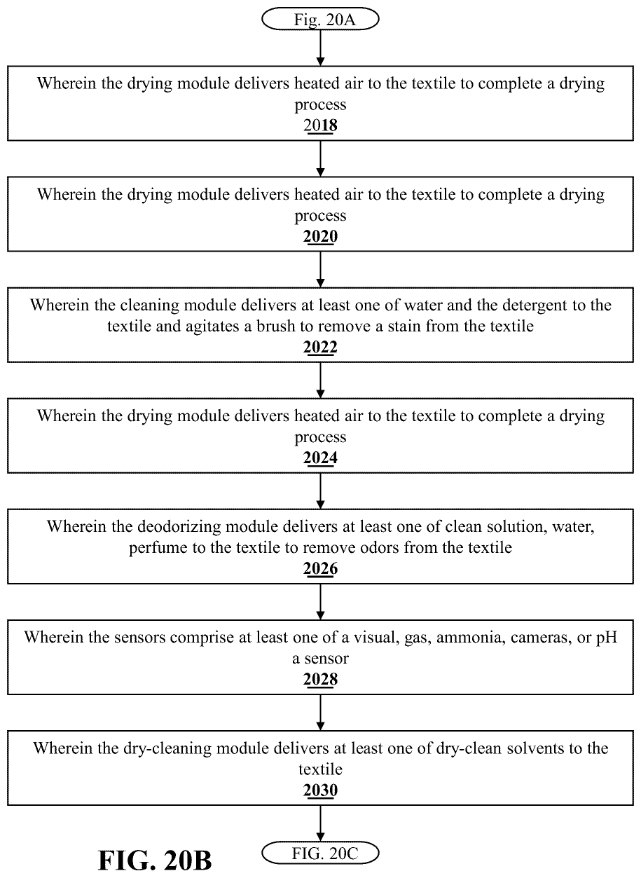

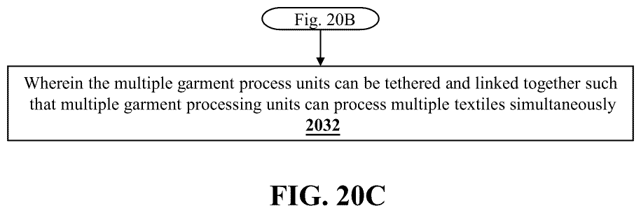

FIG. 20 is a flowchart of an example process 2000. In some implementations, one or more process blocks of FIG. 20 may be performed by a device.

As shown in FIG. 20, process 2000 may include, at 2006, inputting, instructions to the controller to actuate a robot arm to move at least one of a cleaning, a heat source, a deodorizing, a dry-cleaning and a drying module across a surface of a garment. The status signal may be received to input, instructions to the controller to actuate a robot arm to move at least one of a cleaning, a heat source, a deodorizing, a dry-cleaning and a drying module across a surface of a garment, as described above. As also shown in FIG. 20, process 2000 may include delivering, an output, such as perfume to the garment. As further shown in FIG. 20, process 2000 may include, at 2010, causing, application of the outputs to the garment. For example, a device may cause, application of the outputs to the garment, as described above.

Process 2000 may include additional implementations, such as any single implementation or any combination of implementations described below and/or in connection with one or more other processes described elsewhere herein. In the first implementation, at 2012, the outputs are at least one of steam, heat, detergent, dry-cleaning, cleaning solution, perfume and water.

In a second implementation, at 2014, alone or in combination with the first implementation, a sensor is used to indicate with area of the garment needs to be cleaned.

In a third implementation, at 2016, alone or in combination with the first and second implementation, the heat source is attachable to a heat transfer module to cause heat to transfer through the heat transfer module to apply a heat transfer applique to the garment.

In a fourth implementation, at 2018, alone or in combination with one or more of the first through third implementations, the drying module delivers heated air to the garment to complete a drying process.

In a fifth implementation, at 2020, alone or in combination with one or more of the first through fourth implementations, the drying module delivers heated air to the garment to complete a drying process.

In a sixth implementation, at 2022, alone or in combination with one or more of the first through fifth implementations, the cleaning module delivers at least one of water and the detergent to the garment and agitates a brush to remove a stain from the garment.

In a seventh implementation, at 2024, alone or in combination with one or more of the first through sixth implementations, the drying module delivers heated air to the garment to complete a drying process.

In an eighth implementation, at 2026, alone or in combination with one or more of the first through seventh implementations, the deodorizing module delivers at least one of cleaning solution, water, perfume to the garment to remove odors from the garment.

In a ninth implementation, at 2028, alone or in combination with one or more of the first through eighth implementations, the sensors may include at least one of a visual, gas, ammonia, cameras, or pH a sensor.

In a tenth implementation, at 2030, alone or in combination with one or more of the first through ninth implementations, the dry-cleaning module delivers at least one of dry-cleaning solvents to the garment.

In an eleventh implementation, at 2032, alone or in combination with one or more of the first through tenth implementations, the multiple garment processing units can be tethered and linked together such that multiple garment processing units can process multiple garments simultaneously.

Although FIG. 20 shows example blocks of process 2000, in some implementations, process 2000 may include additional blocks, fewer blocks, different blocks, or differently arranged blocks than those depicted in FIG. 20. Additionally, or alternatively, two or more of the blocks of process 2000 may be performed in parallel.

The computing processor can be embodied in the form of hardware, as software components that are executable by hardware, or as a combination of software and hardware. If embodied as hardware, the components described herein can be implemented using one or more processors or processing circuitry, logic circuitry. The hardware can include, one or more microprocessors, discrete logic circuits having logic gates for implementing various logic functions upon an application of one or more data signals, application specific integrated circuits (ASICs) having appropriate logic gates, programmable logic devices (e.g., field-programmable gate array (FPGAs), and complex programmable logic devices (CPLDs)).

If embodied as a combination of software and hardware, the components described herein can be implemented using one or more general-purpose processors or processing circuitry and one or more memory devices. Such processing circuit can include, for example, one or more processors and one or more storage or memory devices that are coupled to a local interface. The local interface can include, for example, a data bus with an accompanying address/control bus or any other suitable bus structure. The storage or memory devices can store data or components that are executable by the processors of the processing circuit.

Although embodiments have been described herein in detail, the descriptions are by way of example. The features of the embodiments described herein are representative and, in alternative embodiments, certain features and elements may be added or omitted. Additionally, modifications to aspects of the embodiments described herein may be made by those skilled in the art without departing from the spirit and scope of the present invention defined in the following claims, the scope of which are to be accorded the broadest interpretation to encompass modifications and equivalent structures.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover all adaptations or variations of various embodiments. Combinations of the above embodiments, and other embodiments not specifically described herein, will be apparent to those of skill in the art upon reviewing the above description.

What is claimed is:

1. A method for controlling a garment processing unit comprising: a memory device;
a controller;
inputting, instructions to the controller to actuate a robot arm to move at least one of a cleaning, a heat source, a deodorizing, a dry-cleaning and a drying module across a surface of a textile;
delivering, outputs to the textile; and
causing, application of the outputs to the textile.

2. The method of claim 1, wherein the outputs are at least one of steam, heat, detergent, dry-cleaning, cleaning solution, perfume and water.

3. The method of claim 2, wherein a sensor is used to indicate with area of the garment needs to be cleaned.

4. The method of claim 2, wherein the heat source is attachable to a heat transfer module to cause heat to transfer through the heat transfer module to apply a heat transfer applique to the garment.

5. The method of claim 2, wherein the cleaning module delivers at least one of water and the detergent to the garment and agitates a brush to remove a stain from the garment.

6. The method of claim 2, wherein the deodorizing module delivers at least one of cleaning solution, water, perfume to the garment to remove odors from the garment.

7. The method of claim 2, wherein the dry-cleaning module delivers at least one of dry-cleaning solvents to the garment.

8. The method of claim 4, wherein the drying module delivers heated air to the garment to complete a drying process.

9. The method of claim 5, wherein the drying module delivers heated air to the garment to complete a drying process.

10. The method of claim 1, wherein the multiple garment processing units can be tethered and linked together such that multiple garment processing units can process multiple garments simultaneously.

11. A device comprising:
one or more processors configured to:
input, instructions to the controller to actuate a robot arm to move at least one of a cleaning, a heat source, a deodorizing, a dry-cleaning and a drying module across a surface of a garment;
deliver, outputs to the garment; and
cause, application of the outputs to the garment.

12. The device of claim 11, wherein the outputs are at least one of steam, heat, detergent, dry-cleaning, cleaning solution, perfume and water.

13. The device of claim 12, wherein a sensor is used to indicate with area of the garment needs to be cleaned.

14. The device of claim 12, wherein the heat source is attachable to a heat transfer module to cause heat to transfer through the heat transfer module to apply a heat transfer applique to the garment.

15. The device of claim 12, wherein the drying module delivers heated air to the garment to complete a drying process.

16. The device of claim 12, wherein the cleaning module delivers at least one of water and the detergent to the garment and agitates a brush to remove a stain from the garment.

17. The device of claim 12, wherein the drying module delivers heated air to the garment to complete a drying process.

18. The device of claim 12, wherein the deodorizing module delivers at least one of cleaning solution, water, perfume to the garment to remove odors from the garment.

19. The device of claim 18, wherein the sensors comprise at least one of a visual, gas, ammonia, cameras, or pH a sensor.

20. The device of claim 12, wherein the dry-cleaning module delivers at least one of dry-cleaning solvents to the garment.
